(19) United States
(12) Patent Application Publication
(10) Pub. No.: US 2026/0035351 A1
(43) Pub. Date: Feb. 5, 2026

(54) SYNTHETIC METHOD OF SULFAMERAZINE

(71) Applicant: Nanhai Beisha Pharmaceutical Co., Ltd., Foshan (CN)

(72) Inventors: Weijie QIU, Foshan (CN); Aichen TANG, Foshan (CN); Weifeng ZHUANG, Foshan (CN); Junwen MO, Foshan (CN); Guangrui HUANG, Foshan (CN)

(21) Appl. No.: 18/976,178

(22) Filed: Dec. 10, 2024

(30) Foreign Application Priority Data
Jul. 31, 2024 (CN) 202411035486.2

(51) Int. Cl. C07D 239/69

(52) U.S. Cl. CPC C07D 239/69

(57) ABSTRACT

Disclosed is a synthetic method of sulfamerazine, relating to the technical field of drug preparation. Acetone and methyl formate are used as initial raw materials, and subjected to condensation reaction to obtain a compound 2; the compound 2 is subjected to addition reaction in a medium of methyl hydrogen sulfate to obtain a compound 3; the compound 3 is subjected to cyclization reaction in a medium of sulfaguanidine to obtain a compound 4; and the compound 4 is subjected to acidification in an acidic medium to obtain the sulfamerazine.

### CROSS REFERENCE TO RELATED APPLICATION

This patent application claims the benefit and priority of Chinese Patent Application No. 202411035486.2 filed with the China National Intellectual Property Administration on Jul. 31, 2024, the disclosure of which is incorporated by reference herein in its entirety as part of the present application.

### TECHNICAL FIELD

The present disclosure relates to the technical field of drug preparation, and in particular to a synthetic method of sulfamerazine.

### BACKGROUND

Sulfonamides are the earliest synthetic antibacterials which are generally white or light yellow crystalline powders. Since their emergence and application in the 1930s, the sulfonamides have been widely valued and studied as the most valuable antibacterial drugs due to easy production and storage, desirable efficacy, convenient use, and low price. Sulfonamides have a history of over 80 years, and over 8,500 sulfonamides have been synthesized, where over 20 types including sulfadiazine, sulfamonomethoxine, sulfamerazine, and sulfamethazine are commonly used in clinical practice. With the continuous discovery and development of various antibiotics, antibiotics and quinolones have gradually replaced the sulfonamides. However, the sulfonamides still exhibit their unique advantages such as wide antibacterial spectrums, stable properties, easy application, low price, and no food consumption in drug production, and mass production. The discoveries of antibacterial enhancers such as trimethoprim (TMP) and diaveridine (DVD) have expanded the antibacterial spectrums and greatly enhanced the antibacterial activities of sulfonamides when being used in combination with the antibacterial enhancers. Therefore, sulfonamides are still one of the most important drugs in the anti-infection treatment of livestock and poultry.

Sulfamerazine, as one type of the sulfonamides, is a white or light yellow crystalline powder that is odorless, has a bitter taste, and changes color on exposure to light. Similar to sulfadiazine, the sulfamerazine is used for infections such as Staphylococcus, Streptococcus hemolyticus, Streptococcus pneumoniae, and Neisseria meningitidis, and has a high efficacy for infections caused by the Streptococcus hemolyticus and Streptococcus pneumoniae. Accordingly, sulfamerazine is also suitable for pneumonia, erysipelas, meningitis and other diseases, and shows the characteristics of rapid absorption and low toxicity.

At present, the synthesis of sulfamerazine is mainly conducted by condensation of 2-amino-4-methylpyrimidine or N-acetylsulfanilyl chloride, with a reaction route as follows:

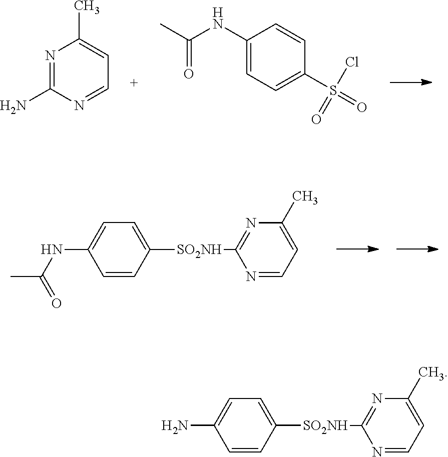

However, the above synthesis method has a low product yield and high raw material cost. Moreover, the N-acetylsulfanilyl chloride is toxic and could enter the human body through inhalation, ingestion, or skin contact, causing respiratory irritation, digestive system discomfort, and poisoning symptoms. As a result, there is an urgent need to have a method for synthesizing sulfamerazine with low cost and high safety.

### SUMMARY

In view of this, an object of the present disclosure is to provide a synthetic method of sulfamerazine with a high yield, low cost, and high safety.

To achieve the above object, the present disclosure provides the following technical solutions:

The present disclosure provides a synthetic method of sulfamerazine, including the following steps:
mixing acetone, methyl formate, and a first sodium alcoholate solution, and subjecting a resulting mixture to condensation reaction, to obtain a reaction mixture of a compound 2;
mixing the reaction mixture of the compound 2 with methyl hydrogen sulfate, adjusting a pH value of an obtained mixture to 1-2, and subjecting a resulting adjusted mixture to addition reaction, to obtain a reaction mixture of a compound 3;
mixing the reaction mixture of the compound 3, sulfaguanidine, and a second sodium alcoholate solution, and subjecting a resulting mixture to cyclization reaction, to obtain a reaction mixture of a compound 4; and
subjecting the reaction mixture of the compound 4 to acidification, to obtain the sulfamerazine; where the compound 2, the compound 3 and the compound 4 are shown as follows:

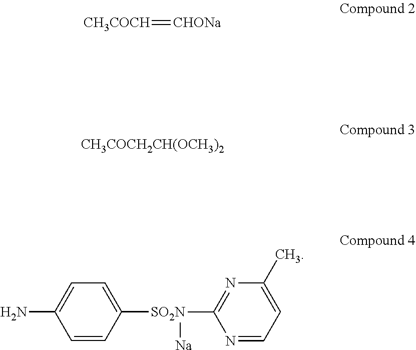

In some embodiments, a mass ratio of the acetone to the methyl formate is in a range of 1:1.3 to 1:2.2;
a mass ratio of the acetone to the first sodium alcoholate solution is in a range of 1:5.9 to 1:33.4; and
a first sodium alcoholate in the first sodium alcoholate solution comprises at least one selected from the group consisting of sodium methoxide, sodium ethoxide, and sodium tert-butoxide, a solvent in the first sodium alcoholate solution includes at least one selected from the group consisting of xylene and toluene, and the first sodium alcoholate solution has a first sodium alcoholate concentration of 5 wt % to 15 wt %.

In some embodiments, the condensation reaction is conducted at a temperature of 50° C. to 60° C. for 3 h to 4 h.

In some embodiments, a mass ratio of the reaction mixture of the compound 2 to the methyl hydrogen sulfate is in a range of 1:0.1 to 1:0.43; and the addition reaction is conducted at a temperature of 30° C. to 45° C. for 3 h to 6 h.

In some embodiments, the method further includes: after the addition reaction, neutralizing a reaction solution obtained by the addition reaction with a sodium methoxide solution, and subjecting a resulting mixture to a first solid-liquid separation to obtain a first liquid component and a first solid component; washing the first solid component to obtain a washing liquor; and combining the first liquid component and the washing liquor and concentrating, to obtain the reaction mixture of the compound 3; where a solvent used for the washing includes at least one selected from the group consisting of xylene and methanol; and a mass ratio of the reaction mixture of the compound 2 to the reaction mixture of the compound 3 is in a range of 1:0.92 to 1:1.03.

In some embodiments, a mass ratio of the reaction mixture of the compound 3 to the sulfaguanidine is in a range of 1:0.15 to 1:0.5;
a mass ratio of the reaction mixture of the compound 3 to the second sodium alcoholate solution is in a range of 1:0.15 to 1:0.5; and
a second sodium alcoholate in the second sodium alcoholate solution includes at least one selected from the group consisting of sodium methoxide, sodium ethoxide, and sodium tert-butoxide, a solvent in the second sodium alcoholate solution includes at least one selected from the group consisting of methanol and ethanol, and the second sodium alcoholate solution has a second sodium alcoholate concentration of 25 wt % to 32 wt %.

In some embodiments, the cyclization reaction is conducted at a temperature of 60° C. to 80° C. for 4 h to 6 h.

In some embodiments, the method further includes: after the cyclization reaction, recovering methanol from a reaction solution obtained by the cyclization reaction, adding water thereto, layering to collect an aqueous layer; subjecting the aqueous layer to cooling crystallization, and subjecting a resulting system to a second solid-liquid separation, to obtain a second liquid component, namely the reaction mixture of the compound 4.

In some embodiments, an acid for the acidification includes at least one selected from the group consisting of hydrochloric acid, sulfuric acid, and acetic acid; and the acidification is conducted to reach a pH value of 6.5 to 7.5.

In some embodiments, the method further includes: after the acidification, subjecting a reaction solution obtained by the acidification to decolorization by adding activated carbon and an alkaline reagent thereto, and subjecting a resulting decolorization system to a third solid-liquid separation to obtain a third liquid component; neutralizing the third liquid component, subjecting a resulting system to a fourth solid-liquid separation to obtain a solid component, and washing the solid component with water and then drying.

Acetone and methyl formate are used as initial raw materials and subjected to condensation reaction to obtain a compound 2; the compound 2 is subjected to addition reaction in a medium of methyl hydrogen sulfate to obtain a compound 3; the compound 3 is subjected to cyclization reaction in a medium of sulfaguanidine to obtain a compound 4; and the compound 4 is subjected to acidification in an acidic medium to obtain the sulfamerazine. The preparation method has the advantages of high product yield and product purity; cheap and readily available raw materials; simple, safe, and stable process with high efficiency and environmental friendliness; mild reaction conditions; simple operations; and convenience for industrial production. Moreover, the synthetic method does not need to separate intermediates, and exhibits simple post-treatment, thereby further reducing a production cost of the sulfamerazine and generating less waste liquid. After acidification and purification of sulfamerazine, the remaining mother liquor could be recovered and reused in the post-treatment after the cyclization to replace the added water, thus further improving a utilization rate of the raw materials to achieve high economic benefits.

### BRIEF DESCRIPTION OF THE DRAWINGS

FIG. 1 shows the 1H NMR spectrum of sulfamerazine prepared in Example 1.

FIG. 2 shows the MS+ spectrum of sulfamerazine prepared in Example 1.

FIG. 3 shows the MS− spectrum of sulfamerazine prepared in Example 1.

FIG. 4 shows the IR spectrum of sulfamerazine prepared in Example 1.

FIG. 5 shows the HPLC spectrum of sulfamerazine prepared in Example 1.

### DETAILED DESCRIPTION OF THE EMBODIMENTS

The present disclosure provides a synthetic method of sulfamerazine, including the following steps:
mixing acetone, methyl formate, and a first sodium alcoholate solution, and subjecting a resulting mixture to condensation reaction, to obtain a reaction mixture of a compound 2;
mixing the reaction mixture of the compound 2 with methyl hydrogen sulfate, adjusting a pH value of an obtained mixture to 1-2, and subjecting a resulting adjusted mixture to addition reaction, to obtain a reaction mixture of a compound 3;
mixing the reaction mixture of the compound 3, sulfaguanidine, and a second sodium alcoholate solution, and subjecting a resulting mixture to cyclization reaction, to obtain a reaction mixture of a compound 4; and
subjecting the reaction mixture solution of the compound 4 to acidification, to obtain the sulfamerazine;
where the compounds 2 to 4 are shown as follows:

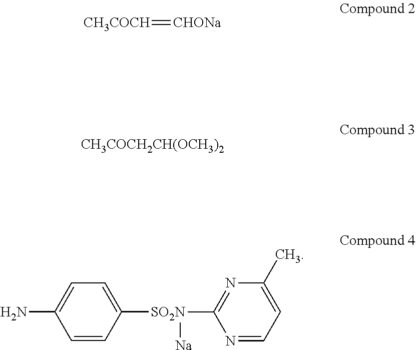

In the present disclosure, unless otherwise specified, all materials and equipment used are commercially available items in the art.

In the present disclosure, acetone, methyl formate, and a first sodium alcoholate solution are mixed and a resulting mixture is subjected to condensation reaction, to obtain a reaction mixture of a compound 2.

In some embodiments of the present disclosure, a mass ratio of the acetone to the methyl formate is in a range of 1:(1.3-2.2), preferably 1:(1.5-2), and more preferably 1:(1.5-1.8).

In some embodiments of the present disclosure, a mass ratio of the acetone to the first sodium alcoholate solution is in a range of 1:(5.9-33.4), preferably 1:(8.3-26.7), and more preferably 1:(9.5-22.2), and most preferably 1:(9.5-17.8).

In some embodiments of the present disclosure, a first sodium alcoholate in the first sodium alcoholate solution includes at least one selected from the group consisting of sodium methoxide, sodium ethoxide, and sodium tert-butoxide, and preferably is sodium methoxide. In some embodiments, a solvent in the first sodium alcoholate solution includes at least one selected from the group consisting of xylene and toluene, and preferably is xylene. In some embodiments, the first sodium alcoholate solution has a first sodium alcoholate concentration of 5 wt % to 15 wt %, preferably 7 wt % to 12 wt %, and more preferably 8 wt % to 10 wt %.

In some embodiments of the present disclosure, the step of mixing the acetone, the methyl formate, and the first sodium alcoholate solution includes: simultaneously adding the methyl formate and the acetone dropwise into the first sodium alcoholate solution; where a solvent in the first sodium alcoholate solution is an organic solvent. In some embodiments, the adding dropwise is conducted at a temperature of 50° C. to 60° C., and preferably 55° C. In some embodiments, the adding dropwise is conducted for 1 h to 2 h, and preferably 1.5 h to 2 h. The condensation reaction occurs during the adding dropwise.

In some embodiments of the present disclosure, the condensation reaction is conducted at a temperature of 50° C. to 60° C., and preferably 55° C. In some embodiments, the condensation reaction is conducted for 3 h to 4 h, and preferably 3.5 h to 4 h (including the time for adding dropwise). In the present disclosure, a reaction solution obtained by the condensation reaction is the mixed solution of the compound 2.

In the present disclosure, after obtaining the mixed solution of the compound 2, the mixed solution of the compound 2 and methyl hydrogen sulfate are mixed, a pH value of an obtained mixture is adjusted to 1-2, and a resulting adjusted mixture is subjected to addition reaction, to obtain the mixed solution of the compound 3.

In some embodiments of the present disclosure, a mass ratio of the mixed solution of the compound 2 to the methyl hydrogen sulfate is in a range of 1:(0.1-0.43), preferably 1:(0.15-0.4), and more preferably 1:(0.2-0.3).

In some embodiments of the present disclosure, the step of mixing the mixed solution of the compound 2 and the methyl hydrogen sulfate includes dropwise adding the mixed solution of the compound 2 into the methyl hydrogen sulfate. In some embodiments, the adding dropwise is conducted at a temperature of 30° C. to 45° C., preferably 35° C. to 40° C. In some embodiments, the adding dropwise is conducted for 1 h to 2 h, and preferably 1.5 h to 2 h.

In some embodiments of the present disclosure, the addition reaction is conducted at a temperature of 30° C. to 45° C., and preferably 35° C. to 40° C. In some embodiments, the addition reaction is conducted for 3 h to 6 h, and preferably 4 h to 5 h (including the time for adding dropwise). In some embodiments, the addition reaction is conducted under stirring.

In some embodiments of the present disclosure, an acid for adjusting the pH value to 1-2 includes at least one selected from the group consisting of hydrochloric acid, sulfuric acid, and acetic acid, and preferably is acetic acid. In the present disclosure, there is no special limitation on a dosage of the acid, as long as the pH value of the obtained mixture could be adjusted to 1-2. In some embodiments, the pH value is adjusted to 1-1.5.

In some embodiments of the present disclosure, the method further includes: after the addition reaction, neutralizing a reaction solution obtained by the addition reaction with a sodium alcoholate solution (denoted as a third sodium alcoholate solution), and subjecting a resulting mixture to solid-liquid separation to obtain a liquid component and a solid component; washing the solid component to obtain a washing liquor; and combining the liquid component and the washing liquor and concentrating to obtain the mixed solution of the compound 3; where a solvent for the washing includes at least one selected from the group consisting of xylene and methanol.

In some embodiments of the present disclosure, a third sodium alcoholate in the third sodium alcoholate solution includes at least one selected from the group consisting of sodium methoxide, sodium ethoxide, and sodium tert-butoxide, and preferably is sodium methoxide. In some embodiments, a solvent in the third sodium alcoholate solution includes at least one selected from the group consisting of methanol and ethanol, and preferably is methanol. In some embodiments, the third sodium alcoholate solution has a third sodium alcoholate concentration of 25 wt % to 32 wt %, preferably 25 wt % to 30 wt %, and more preferably 25 wt % to 28 wt %. In the present disclosure, there is no particular limitation on a dosage of the third sodium alcoholate solution, as long as the system could be neutralized to a pH value of 6.8-7.5. In some embodiments, the pH value is adjusted to 7-7.2.

In the present disclosure, there is no particular limitation on the solid-liquid separation, and any means for the solid-liquid separation well known to those skilled in the art may be used, such as filtration, suction filtration, or centrifugation.

In some embodiments of the present disclosure, the concentration includes recovering methanol at atmospheric pressure, water separation under reduced pressure, and distillation under reduced pressure, which are conducted sequentially.

In some embodiments of the present disclosure, a mass ratio of the mixed solution of the compound 2 to the mixed solution of the compound 3 obtained by concentration is in a range of 1:(0.92-1.03), and preferably 1:(0.98-1).

In the present disclosure, after obtaining the mixed solution of the compound 3, the mixed solution of the compound 3, sulfaguanidine, and a second sodium alcoholate solution are mixed, and a resulting mixture is subjected to cyclization reaction, to obtain a mixed solution of a compound 4.

In some embodiments of the present disclosure, a mass ratio of the mixed solution of the compound 3 to the sulfaguanidine is in a range of 1:(0.15-0.5), preferably 1:(0.15-0.3), and more preferably 1:(0.15-0.2).

In some embodiments of the present disclosure, a second sodium alcoholate in the second sodium alcoholate solution includes at least one selected from the group consisting of sodium methoxide, sodium ethoxide, and sodium tert-butoxide, and preferably sodium methoxide. In some embodiments, a solvent in the second sodium alcoholate solution includes at least one selected from the group consisting of methanol and ethanol, and preferably methanol. In some embodiments, the second sodium alcoholate solution has a second sodium alcoholate concentration of 25 wt % to 32 wt %, preferably 25 wt % to 30 wt %, and more preferably 25 wt % to 28 wt %.

In some embodiments of the present disclosure, a mass ratio of the mixed solution of the compound 3 to the second sodium alcoholate solution is in a range of 1:(0.15-0.5), preferably 1:(0.15-0.3), and more preferably 1:(0.15-0.2).

In some embodiments of the present disclosure, the step of mixing the mixed solution of the compound 3, sulfaguanidine and the second sodium alcoholate solution includes: heating sulfaguanidine and the second sodium alcoholate solution to reflux, and then adding dropwise the reaction mixture of the compound 3. In some embodiments, the adding dropwise of the reaction mixture of the compound 3 is conducted within 0.5 h to 3 h, preferably 1 h to 2.5 h, and more preferably 1.5 h to 2 h. The cyclization reaction occurs during the adding dropwise.

In some embodiments of the present disclosure, the cyclization reaction is conducted at a temperature of 60° C. to 80° C., preferably 65° C. to 75° C., and more preferably 70° C. In some embodiments, the cyclization reaction is conducted for 4 h to 6 h, preferably 4.5 h to 5.5 h, and more preferably 5 h (including the time for adding dropwise).

In some embodiments of the present disclosure, the method further includes: after the cyclization reaction, recovering methanol from a reaction solution obtained by the cyclization reaction, adding water thereto, layering and separating an aqueous layer, subjecting the aqueous layer to cooling crystallization, and subjecting a resulting system to solid-liquid separation to obtain a liquid component, namely the reaction mixture of the compound 4.

In some embodiments of the present disclosure, the methanol is recovered at a temperature of 60° C. to 80° C., preferably 65° C. to 75° C., and more preferably 70° C. Recovering alcohol solvents (methanol and/or ethanol) under the above conditions could ensure that the alcohol solvents are completely recovered while reducing the carryover of organic solvents such as xylene. Since methanol and ethanol are soluble in water, failure to completely recover them could affect the layering of water and organic solvents such as xylene after adding the water, thereby adversely affecting a product yield.

In some embodiments of the present disclosure, the cooling crystallization is conducted at a temperature of 25° C. to 30° C. In some embodiments, the cooling crystallization is conducted for 3 h to 4 h, and preferably 3 h to 3.5 h.

In some embodiments of the present disclosure, there is no particular limitation on the solid-liquid separation, and any means for the solid-liquid separation well known to those skilled in the art may be used, such as filtration, suction filtration, or centrifugation.

In some embodiments of the present disclosure, a solid component is obtained by the solid-liquid separation, and the solid component is dried to obtain sulfaguanidine, and the sulfaguanidine could be recycled.

In the present disclosure, after obtaining the reaction mixture of the compound 4, the reaction mixture of the compound 4 is subjected to acidification to obtain the sulfamerazine.

In some embodiments of the present disclosure, an acid for the acidification includes at least one selected from the group consisting of hydrochloric acid, sulfuric acid, and acetic acid, and preferably is acetic acid. There is no particular limitation on a dosage of the acid, as long as the pH value of the system could be adjusted to 6.5-7.5. In some embodiments, the acidification is conducted to reach a pH value of 6.5-7.5, and preferably 6.8-7.

In some embodiments of the present disclosure, the method further includes: after the acidification, subjecting a reaction solution obtained by the acidification to decolorization by adding activated carbon and an alkaline reagent thereto, and subjecting a resulting mixture to solid-liquid separation to obtain a liquid component, neutralizing the liquid component, subjecting a resulting system to solid-liquid separation to obtain a solid component, and washing the solid component with water and then drying.

In some embodiments of the present disclosure, a mass ratio of the reaction mixture of the compound 4 to the activated carbon is in a range of 1:(0.001-0.02), and preferably 1:(0.003-0.01).

In some embodiments of the present disclosure, the alkaline reagent includes at least one selected from the group consisting of lime, sodium hydroxide, and potassium hydroxide, and preferably is lime. In some embodiments, a mass ratio of the reaction mixture of the compound 4 to the alkaline reagent is in a range of 1:(0.001-0.02), and preferably 1:(0.003-0.01).

In some embodiments of the present disclosure, the decolorization is conducted at a temperature of 85° C. to 95° C., preferably 90° C. In some embodiments, the decolorization is conducted for 30 min to 60 min, and preferably 40 min to 50 min.

In the present disclosure, there is no particular limitation on the solid-liquid separation, and any means for solid-liquid separation well known to those skilled in the art may be used, such as filtration, suction filtration, or centrifugation.

In some embodiments of the present disclosure, the neutralization is conducted at a temperature of 80° C. to 90° C., and preferably 85° C. In some embodiments, an acid for the neutralization includes at least one selected from the group consisting of hydrochloric acid, sulfuric acid, and acetic acid, and preferably is acetic acid. In the present disclosure, there is no particular limitation on a dosage of the acid, as long as the pH value of the system could be adjusted to 6.5-7.5. In some embodiments, the pH value is adjusted to 6.8-7.

In some embodiments of the present disclosure, the drying is conducted at a temperature of 90° C. to 120° C., and preferably 95° C. to 105° C. There is no particular limitation on a drying time, as long as drying to constant weight.

To further illustrate the present disclosure, the method for synthesizing sulfamerazine according to the present disclosure is described in detail below in conjunction with examples, but these examples should not be construed as limiting the claimed scope of the present disclosure.

In the following examples, an 8 wt % solution of sodium methoxide in xylene was prepared as follows: 4 kg of a 25 wt % sodium methoxide solution in methanol was weighed, methanol therein was evaporated, 11.5 kg of xylene was then added thereto, a resulting mixture was stirred for 30 min, and then allowed to stand, to obtain the 8 wt % sodium methoxide solution in xylene.

### Example 1

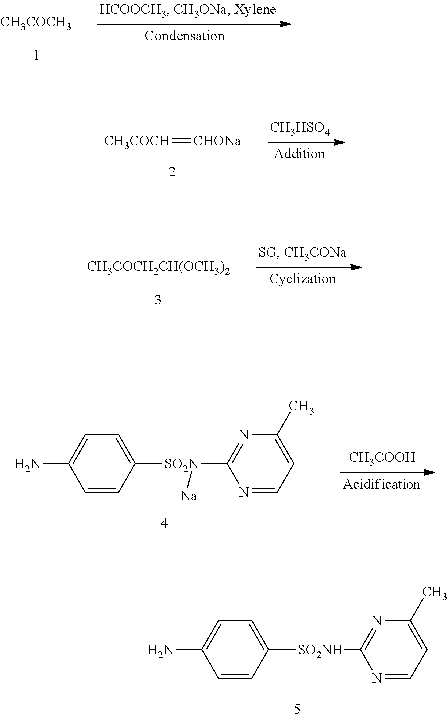

S1-Condensation: methyl formate (1.5 kg) and acetone (1.0 kg) were simultaneously added dropwise into an 8 wt % solution (12.5 kg) of sodium methoxide in xylene at 25° C. within 2 h, and a resulting mixture was heated to 55° C. and reacted for 4 h, to obtain a reaction mixture of a compound 2 (about 14.8 kg).

S2-Addition: the reaction mixture of the compound 2 prepared in step S1 was evenly added dropwise into methyl hydrogen sulfate (3.5 kg) at 35° C. within 2 h, and a resulting mixture was stirred and reacted at pH=1 and below 40° C. for 4 h. A resulting reaction mixture was neutralized with a 25 wt % solution of sodium methoxide in methanol to pH=7.1. A resulting mixture was filtered to obtain a filter cake and a filtrate. The filter cake was washed with xylene (4.5 kg), a resulting washing liquor and filtrate were combined, and methanol was recovered therefrom at atmospheric pressure, followed by water separation under reduced pressure, and distillation under reduced pressure, to obtain a reaction mixture of a compound 3 in xylene (14.5 kg).

S3-Cyclization: a 25 wt % solution (2.5 kg, sodium methoxide concentration being 25 wt %) of sodium methoxide in methanol and sulfaguanidine (2.23 kg) were heated to reflux, and then the reaction mixture of compound 3 in xylene prepared in step S2 was added dropwise thereto within 1.5 h, and all the methanol was recovered therefrom under atmospheric pressure. 25 kg of water was added thereto at 70° C., and a resulting system was layered to obtain an aqueous layer. The aqueous layer was cooled to 25° C. and crystallized for 4 h. A resulting mixture was filtered, and the sulfaguanidine was recovered from a filter cake while a filtrate was a reaction mixture of a compound 4.

S4-Acidification: 0.15 kg of activated carbon and 0.15 kg of lime were added into the reaction mixture of the compound 4 prepared in step S3, and a resulting mixture was heated to 90° C. and subjected to decolorization for 1 h. A resulting decolorization system was filtered, and a filtrate was heated to a temperature of 80° C. to 90° C. Acetic acid was added dropwise thereto to neutralize the filtrate to pH-6.8. A resulting mixture was filtered, a filter cake was washed with clear water, and then dried at 100° C. to a constant weight, to obtain sulfamerazine (i.e., compound 5, 2.6 kg, a total yield of 57%, with a HPLC purity of 99.7% based on acetone).

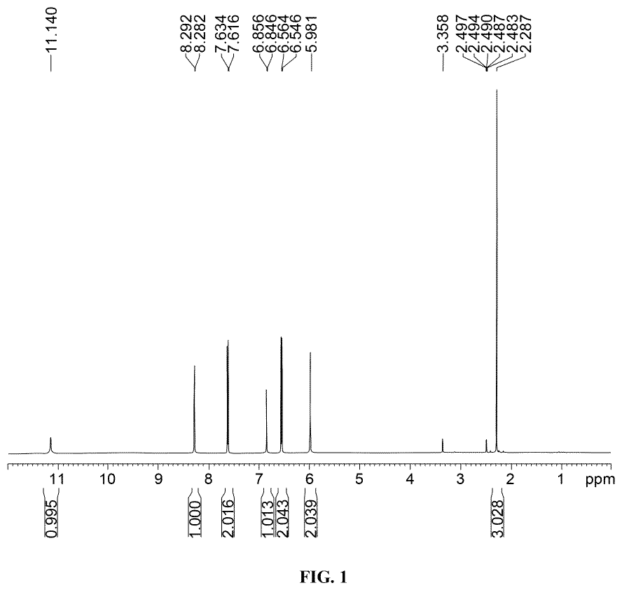

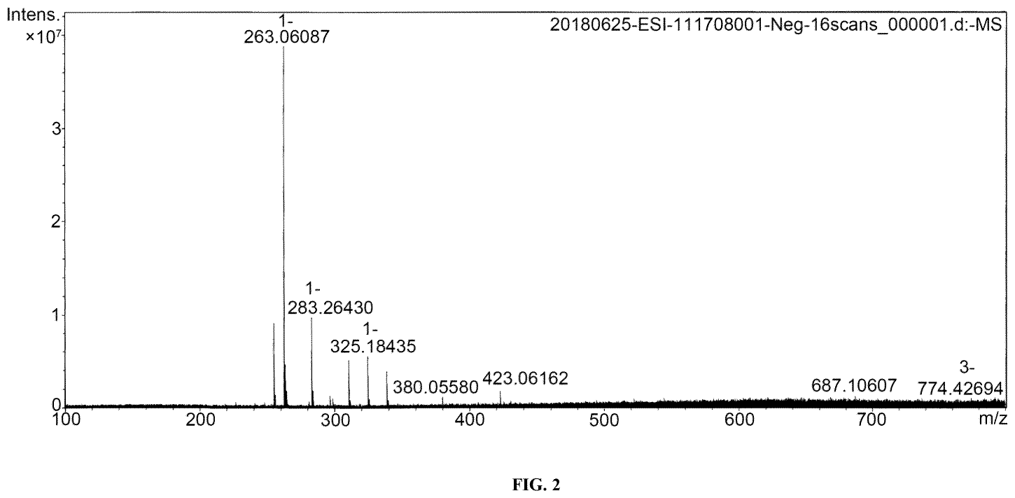

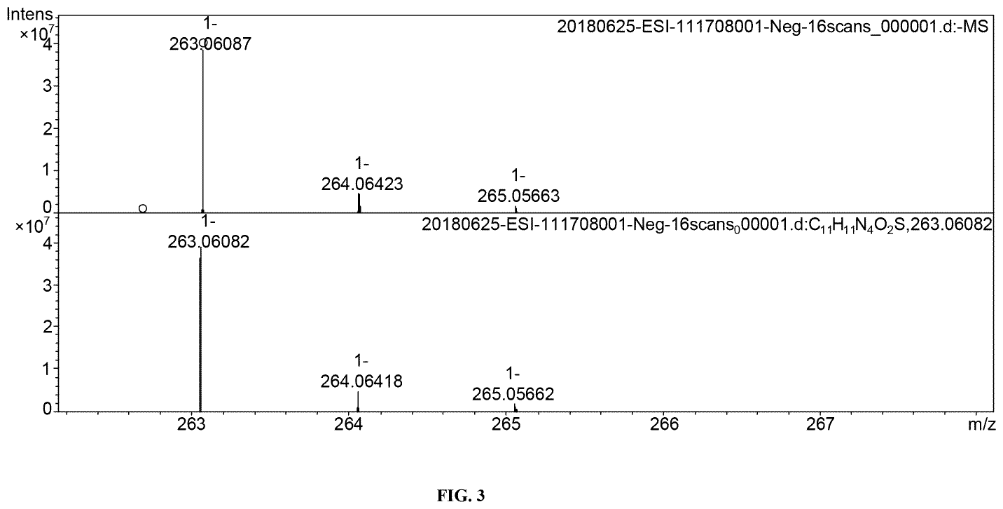

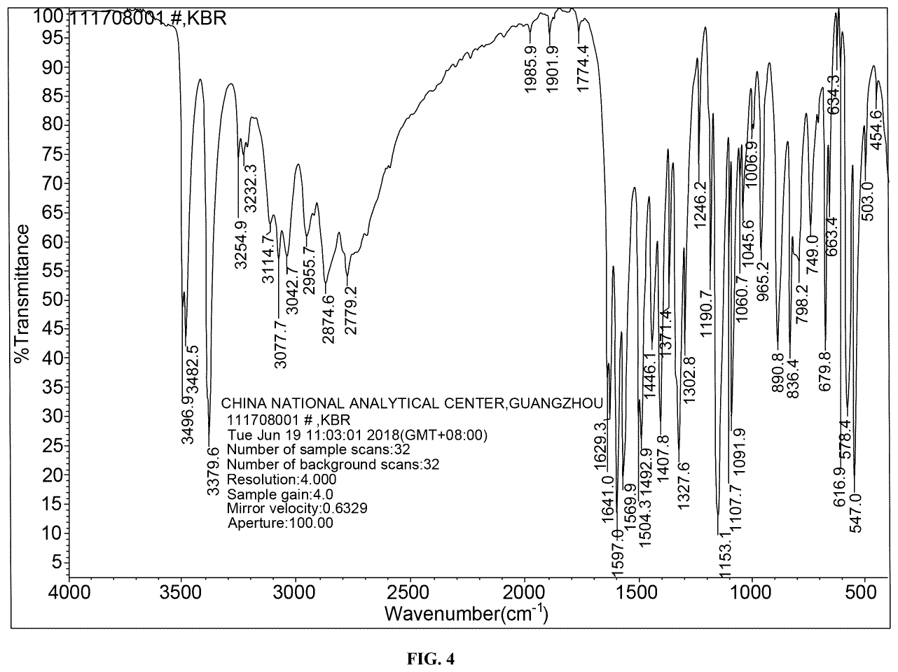

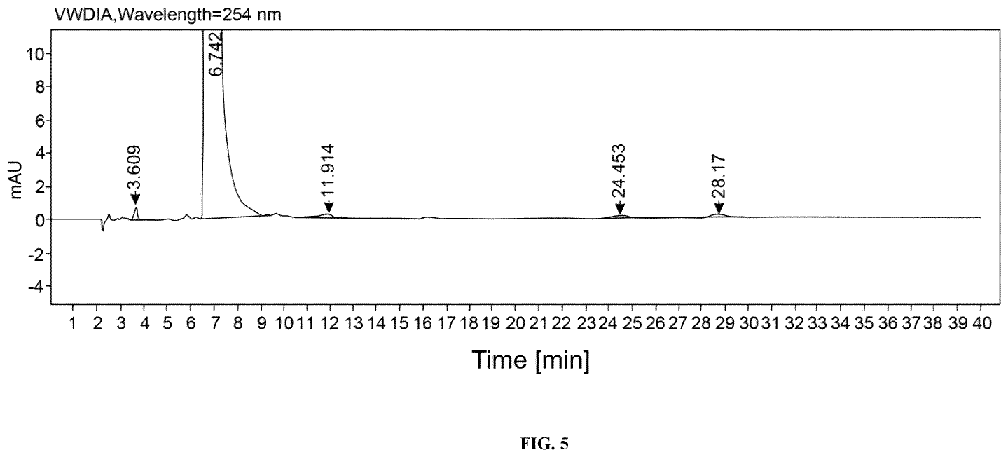

FIG. 1 shows the 1H NMR spectrum of sulfamerazine prepared in Example 1; FIG. 2 shows the MS+ spectrum of sulfamerazine prepared in Example 1; FIG. 3 shows the MS− spectrum of sulfamerazine prepared in Example 1; FIG. 4 shows the IR spectrum of sulfamerazine prepared in Example 1; and FIG. 5 shows the HPLC spectrum of sulfamerazine prepared in Example 1. The chromatographic peak information of the HPLC spectrum is shown in Table 1.

TABLE 1
Chromatographic peak information of HPLC spectrum

| | | Peak | | | Peak | | | |
|---|---|---|---|---|---|---|---|---|
| Retention | | width | Peak area | Height | area | | Tailing | Theoretical |
| time (min) | Type | (min) | (mAU · min) | (mAU) | (%) | Resolution | factor | plate number |
| 3.609 | BV | 0.47 | 6.73588 | 0.91 | 0.050 | | 1.2 | 5772 |
| 6.742 | VV | 3.05 | 13329.82708 | 1066.43 | 99.741 | 12.5 | 1.2 | 7594 |
| 11.914 | BB | 1.63 | 11.34326 | 0.24 | 0.085 | 6.9 | 0.6 | 1625 |
| 24.453 | BB | 2.12 | 7.09576 | 0.16 | 0.053 | 10.9 | 1.2 | 7518 |
| 28.710 | BB | 2.15 | 9.50506 | 0.21 | 0.071 | 3.6 | 1.5 | 8620 |

| | | |
|---|---|---|
| Total | 13364.50703 | 100% |

### Example 2

S1-Condensation: methyl formate (15 kg) and acetone (10 kg) were simultaneously added dropwise into an 8 wt % solution (125 kg) of sodium methoxide in xylene at 25° C. within 2 h, and a resulting mixture was heated to 55° C. and reacted for 4 h, to obtain a reaction mixture of a compound 2 (about 148 kg).

S2-Addition: the reaction mixture of the compound 2 prepared in step S1 was evenly added dropwise into a reactor containing methyl hydrogen sulfate (35 kg) at 35° C. within 2 h, and a resulting mixture was stirred and reacted at pH=1 and below 40° C. for 4 h. A resulting reaction mixture was neutralized with a 25 wt % solution of sodium methoxide in methanol to pH=7.1. A resulting mixture was filtered to obtain a filter cake and a filtrate. The filter cake was washed with xylene (45 kg), the resulting washing liquor and the filtrate were combined, and methanol was recovered therefrom at atmospheric pressure, followed by water separation under reduced pressure, and distillation under reduced pressure, to obtain a reaction mixture of a compound 3 in xylene (145 kg).

S3-Cyclization: a 25 wt % solution (25 kg) of sodium methoxide in methanol and sulfaguanidine (22.3 kg) were heated to reflux, and then 145 kg of the reaction mixture of the compound 3 in xylene was added dropwise thereto within 1.5 h, and all the methanol was recovered therefrom under atmospheric pressure until the temperature reached 70° C. 250 kg of water was added thereto, and a resulting system was layered to obtain an aqueous layer. The aqueous layer was cooled to 25° C. and crystallized for 4 h. A resulting mixture was filtered, and the sulfaguanidine was recovered from a filter cake while a filtrate was a reaction mixture of a compound 4.

S4-Acidification: 1.5 kg of activated carbon and 1.5 kg of lime were added into the reaction mixture of the compound 4 prepared in step S3, and a resulting mixture was heated to 90° C. and subjected to decolorization for 1 h. A resulting decolorization system was filtered, and a filtrate was heated to a temperature of 80° C. to 90° C. Acetic acid was added dropwise thereto to neutralize the filtrate to pH=6.8. A resulting mixture was filtered, and a filter cake was washed with clear water, and then dried at 100° C. to a constant weight, to obtain sulfamerazine (26 kg, a total yield of 57%, with a HPLC purity of 99.7% based on acetone).

### Example 3

S1-Condensation: methyl formate (75 kg) and acetone (50 kg) were simultaneously added dropwise into an 8 wt % solution (625 kg) of sodium methoxide in xylene at 25° C. within 2 h, and a resulting mixture was heated to 55° C. and reacted for 4 h, to obtain a reaction mixture of a compound 2 (about 740 kg).

S2-Addition: the reaction mixture of the compound 2 prepared in step S1 was evenly added dropwise into a reactor containing methyl hydrogen sulfate (175 kg) at 35° C. within 2 h, and a resulting mixture was stirred and reacted at pH=1 and below 40° C. for 4 h. A resulting reaction mixture was neutralized with a 25 wt % solution of sodium methoxide in methanol to pH=7.1. A resulting mixture was filtered to obtain a filter cake and a filtrate. The filter cake was washed with xylene (225 kg), the resulting washing liquor and the filtrate were combined, and methanol was recovered therefrom at atmospheric pressure, followed by water separation under reduced pressure, and distillation under reduced pressure, to obtain a reaction mixture of a compound 3 in xylene (725 kg).

S3-Cyclization: a 25 wt % solution (125 kg) of sodium methoxide in methanol and sulfaguanidine (111.5 kg) were heated to reflux, and then the reaction mixture of the compound 3 in xylene prepared in step S2 was added dropwise thereto within 1.5 h, and all the methanol was recovered therefrom under atmospheric pressure until the temperature reached 70° C. 1,250 kg of water was added thereto, a resulting system was layered to obtain an aqueous layer. The aqueous layer was cooled to 25° C., and crystallized for 4 h. A resulting mixture was filtered, and the sulfaguanidine was recovered from a filter cake while a filtrate was a reaction mixture of a compound 4.

S4-Acidification: 7.5 kg of activated carbon and 7.5 kg of lime were added into the reaction mixture of the compound 4 prepared in step S3, and a resulting mixture was heated to 90° C. and subjected to decolorization for 1 h. A resulting decolorization system was filtered, and a filtrate was heated to a temperature of 80° C. to 90° C. Acetic acid was added dropwise thereto to neutralize the filtrate to pH=6.8. A resulting mixture was filtered, a filter cake was washed with clear water, and then dried at 100° C. to a constant weight, to obtain sulfamerazine (130 kg, a total yield of 57%, with a HPLC purity of 99.7% based on acetone).

The above descriptions are merely preferred embodiments of the present disclosure. It should be noted that a person of ordinary skill in the art may further make several improvements and modifications without departing from the principle of the present disclosure, but such improvements and modifications should be deemed as falling within the scope of the present disclosure.

What is claimed is:

1. A synthetic method of sulfamerazine, comprising:
mixing acetone, methyl formate, and a first sodium alcoholate solution, and subjecting a resulting mixture to condensation reaction, to obtain a reaction mixture of a compound 2;
mixing the reaction mixture of the compound 2 with methyl hydrogen sulfate, adjusting a pH value of an obtained mixture to 1-2, and subjecting a resulting adjusted mixture to addition reaction, to obtain a reaction mixture of a compound 3;
mixing the reaction mixture of the compound 3, sulfaguanidine, and a second sodium alcoholate solution, and subjecting a resulting mixture to cyclization reaction, to obtain a reaction mixture of a compound 4; and
subjecting the reaction mixture of the compound 4 to acidification, to obtain the sulfamerazine;
wherein the compound 2, the compound 3, and the compound 4 are shown as follows:

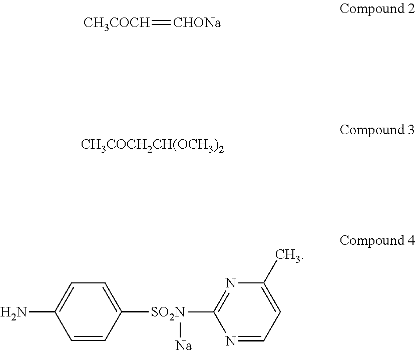

2. The synthetic method as claimed in claim 1, wherein a mass ratio of the acetone to the methyl formate is in a range of 1:1.3 to 1:2.2;
a mass ratio of the acetone to the first sodium alcoholate solution is in a range of 1:5.9 to 1:33.4; and
a first sodium alcoholate in the first sodium alcoholate solution comprises at least one selected from the group consisting of sodium methoxide, sodium ethoxide, and sodium tert-butoxide, a solvent in the first sodium alcoholate solution comprises at least one selected from the group consisting of xylene and toluene, and the first sodium alcoholate solution has a first sodium alcoholate concentration of 5 wt % to 15 wt %.

3. The synthetic method as claimed in claim 1, wherein the condensation reaction is conducted at a temperature of 50° C. to 60° C. for 3 h to 4 h.

4. The synthetic method as claimed in claim 2, wherein the condensation reaction is conducted at a temperature of 50° C. to 60° C. for 3 h to 4 h.

5. The synthetic method as claimed in claim 1, wherein a mass ratio of the reaction mixture of the compound 2 to the methyl hydrogen sulfate is in a range of 1:0.1 to 1:0.43; and
the addition reaction is conducted at a temperature of 30° C. to 45° C. for 3 h to 6 h.

6. The synthetic method as claimed in claim 1, further comprising
after the addition reaction, neutralizing a reaction solution obtained by the addition reaction with a sodium methoxide solution, and subjecting a resulting mixture to a first solid-liquid separation to obtain a first liquid component and a first solid component;
washing the first solid component to obtain a washing liquor; and
combining the first liquid component and the washing liquor, and concentrating, to obtain the reaction mixture of the compound 3; wherein a solvent for the washing comprises at least one selected from the group consisting of xylene and methanol; and a mass ratio of the reaction mixture of the compound 2 to the reaction mixture of the compound 3 is in a range of 1:0.92 to 1:1.03.

7. The synthetic method as claimed in claim 5, further comprising
after the addition reaction, neutralizing a reaction solution obtained by the addition reaction with a sodium methoxide solution, and subjecting a resulting mixture to a first solid-liquid separation to obtain a first liquid component and a first solid component;
washing the first solid component to obtain a washing liquor; and
combining the first liquid component and the washing liquor, and concentrating, to obtain the reaction mixture of the compound 3; wherein a solvent for the washing comprises at least one selected from the group consisting of xylene and methanol; and a mass ratio of the reaction mixture of the compound 2 to the reaction mixture of the compound 3 is in a range of 1:0.92 to 1:1.03.

8. The synthetic method as claimed in claim 1, wherein a mass ratio of the reaction mixture of the compound 3 to the sulfaguanidine is in a range of 1:0.15 to 1:0.5;
a mass ratio of the reaction mixture of the compound 3 to the second sodium alcoholate solution is in a range of 1:0.15 to 1:0.5; and
a second sodium alcoholate in the second sodium alcoholate solution comprises at least one selected from the group consisting of sodium methoxide, sodium ethoxide, and sodium tert-butoxide, a solvent in the second sodium alcoholate solution comprises at least one selected from the group consisting of methanol and ethanol, and the second sodium alcoholate solution has a second sodium alcoholate concentration of 25 wt % to 32 wt %.

9. The synthetic method as claimed in claim 1, wherein the cyclization reaction is conducted at a temperature of 60° C. to 80° C. for 4 h to 6 h.

10. The synthetic method as claimed in claim 8, wherein the cyclization reaction is conducted at a temperature of 60° C. to 80° C. for 4 h to 6 h.

11. The synthetic method as claimed in claim 9, further comprising
after the cyclization reaction, recovering methanol from a reaction solution obtained by the cyclization reaction, adding water thereto, and layering to collect an aqueous layer; and
subjecting the aqueous layer to cooling crystallization, and subjecting a resulting system to a second solid-liquid separation, to obtain a second liquid component, namely the reaction mixture of the compound 4.

12. The synthetic method as claimed in claim 1, wherein an acid for the acidification comprises at least one selected from the group consisting of hydrochloric acid, sulfuric acid, and acetic acid; and
the acidification is conducted to reach a pH value of 6.5 to 7.5.

13. The synthetic method as claimed in claim 1, further comprising
after the acidification, subjecting a reaction solution obtained by the acidification to decolorization by adding activated carbon and an alkaline reagent thereto, and subjecting a resulting decolorization system to a third solid-liquid separation to obtain a third liquid component;
neutralizing the third liquid component, subjecting a resulting system to a fourth solid-liquid separation to obtain a solid component, and washing the solid component with water and then drying.

14. The synthetic method as claimed in claim 12, further comprising
after the acidification, subjecting a reaction solution obtained by the acidification to decolorization by adding activated carbon and an alkaline reagent thereto, and subjecting a resulting decolorization system to a third solid-liquid separation to obtain a third liquid component;
neutralizing the third liquid component, subjecting a resulting system to a fourth solid-liquid separation to obtain a solid component, and washing the solid component with water and then drying.
